(19) United States
(12) Patent Application Publication
(10) Pub. No.: US 2014/0135375 A1
(43) Pub. Date: May 15, 2014

(54) Edible Transgenic Plants as Oral Delivery Vehicles for RNA-Based Therapeutics

(71) Applicant: Eric Lam, Monroe, NJ (US)

(72) Inventor: Eric Lam, Monroe, NJ (US)

(73) Assignee: Rutgers, The State University of New Jersey, New Brunswick, NJ (US)

(21) Appl. No.: 14/006,223

(22) PCT Filed: Apr. 2, 2012
(86) PCT No.: PCT/US12/31830

§ 371 Date: Jan. 30, 2014

Related U.S. Application Data
(60) Provisional application No. 61/470,717, filed on Apr. 1, 2011.

(51) Int. Cl. A61K 36/81; A61K 31/7088

(52) U.S. Cl. CPC A61K 36/81; A61K 31/7088
USPC 514/44 A; 800/298; 800/317.4; 800/317.3; 800/317.2; 800/320.2; 800/320.1; 800/307

(57) ABSTRACT

Compositions and methods for delivery of therapeutic RNA molecules are disclosed.

This application claims priority to U.S. Provisional Application 61/470,717 filed Apr. 1, 2011, the entire contents being incorporated herein by reference as though sent forth in full.

### FIELD OF THE INVENTION

The present invention relates to the fields of transgenic plants and control of target gene expression. More specifically, the present invention provides compositions and methods for the production of edible plants expressing RNAi effective to downregulate important therapeutic targets. Such plants, their derivatives, seeds or progeny can be ingested for the prevention and/or treatment of disease or infection.

### BACKGROUND OF THE INVENTION

Several publications and patent documents are cited throughout the specification in order to describe the state of the art to which this invention pertains. Each of these citations is incorporated herein by reference as though set forth in full.

Viruses such as hepatitis C virus (HCV), avian influenza (H5N1) and the human immunodeficiency virus (HIV) pose difficult targets for traditional therapies and preventive measures. Their rapid mutation and recombination rates make monotherapeutics involving single targets ineffective in the long term. Although RNA interference (RNAi) triggered by administration or expression of small double-stranded RNAs (dsRNAs) of 21-25 nucleotides targeting essential components of various viruses has been shown to be effective in suppressing their replication in host cells, spontaneous mutations in the targeted virus can evolve to escape this repression. In addition, the ultimate deployment of RNAi as an antiviral therapeutics in the developing world will likely be hampered by the economics involved. Thus, the process by which such dsRNA molecules can be produced and delivered to people who need them most, such as places that are high risk for AIDS, may be another critical factor. Clearly, compositions and methods which are effective for deployment of RNAi in vivo for a variety of therapeutic applications are urgently needed.

### SUMMARY OF THE INVENTION

In accordance with the present invention, compositions and methods are provided for the delivery of small RNA molecules in vivo for the treatment of infection or disease. In one embodiment, transgenic plants, e.g., tomato, tobacco, carrot, lettuce, potato, rice, corn, cucumber and zucchini), comprising a nucleic acid construct encoding at least one inhibitory RNA molecule are disclosed. In another embodiment, 2, 3, 4 or 5 inhibitory RNA molecules can be introduced into said plant. Exemplary RNAi for use in the invention include those effective against certain viral pathogens which include, but are not limited to human immunodeficiency virus, hepatitis C virus, influenza virus.

In yet another aspect, a method for inhibiting viral infection or disease in a mammal is disclosed. An exemplary method entails ingestion of an effective amount of the transgenic plant or fruit of the invention by said mammal, said plant or fruit expressing at least one nucleic acid construct encoding an inhibitory RNA, said inhibitory RNA being effective to inhibit viral infection, and/or replication or disease in said mammal

Also encompassed by the present invention are transgenic plants and fruits which express RNAi of interest. Also provided are seeds and progeny of such plants.

### BRIEF DESCRIPTION OF THE DRAWINGS

FIG. 1. Outline of steps and vectors for testing plant-derived siRNA production in tomato as delivery vehicle via ingestion. a) Overall flow of the strategy using rabbits as the model mammal to test uptake and stability of plant derived RNAs via ingestion. Diagram on top illustrates the relevant part of the binary vector pEL420 used for production of siRNAs against different animal viruses as indicated. b) Viral sequences (51 nucleotides per line) used for dsRNA production that were cloned individually into EL420 vector to produce the vectors HIVi, NPi and HCVi as indicated. c) Binary vector structure of pEL420 for dsRNA production in planta. NPTII encodes the Kanamycin selection marker for transformation while the GusA gene encodes the reporter gene for β-glucuronidase. p35S is the constitutive 35S promoter from cauliflower mosaic virus; I is the intron-2 from the Pdk gene of Flaveria that is placed between two inverted copies (indicated by two tail-to-tail arrows in panel a) of the viral sequence; Ter: a polyA addition and transcription terminator sequence from the octapine synthase gene of Agrobacterium (Tocs); ccdB: bacterial counter-selection gene where desired sequence for insertion will replace via the recombinase target att sites; WT: wild-type plant.

FIG. 2. GUS expression in transgenic tomato lines. Biochemical assays with the fluorigenic substrate 4-MUG were carried out with extracts from mature leaves, green fruits and red fruits from 12 selected lines. Specific activity normalized to mg protein per hr is shown.

FIG. 3. Northern blot detection of sRNAs from tomato fruit and leaf tissues. sRNA enriched RNAs are prepared and separated on a 15% PAGE before transfer to hybond-N+ membranes. The two blots containing fruit and leaf RNAs are each blotted sequentially with radiolabelled HCV NP gene probe or tomato miRNA159 oligonucleotide probe. P: putative unprocessed precursor to siRNAs; si: siRNAs; mi: miRNA. Wild type (wt) and transgenic lines are as indicated.

FIG. 4. Detection and quantification of steady state tomato transcripts and sRNAs in tomatoes. TaqMan probes synthesized by InVitrogen/Applied Biosystems are used to quantify transcripts in RNA preparations from tomato fruits. A. Nuclear- and plastid-encoded transcripts chosen for quantification in tomato fruits. 16S rRNA, RbcL and RpoC1 are plastid-encoded genes with varying levels of expression while TomUbi3 encodes a ubiquitin gene of tomato while ACCS encodes an enzyme involved in ethylene synthesis and is fruit-specific. Levels of each transcript is normalized to that of RpoC1, which expresses at the lowest level among the 5 transcripts. Total RNA was used for RT-PCR. B. The levels of siRNAs for each of the 3 viral sequences are quantified by KLP-TaqMan in transgenic tomato fruits, using tomato miRNA164 as a common reference. sRNA enriched RNA preparations are used for cDNA synthesis. WT (wild-type) tomato RNAs do not show any significant levels of amplification with the 3 KLP probes, although only data for the HCVi probe is shown. The lines used are indicated on the X-axis. Note the scale on the Y-axis for both panels is in Log 10 since there is a large range of variations in expression levels.

FIG. 5. Detection of tomato transcripts in the blood and tissues of rabbits after ingestion of ripened fruits in the diet. Rabbits of ˜1.8 kg were fed for 14 days with either normal chow (Control) or a 1:1 mix (w/w) of diced tomato+chow (Tomato). Panel A: detection of transcripts from tomato in the blood of rabbits post-ingestion by RT-PCR followed by re-amplification with a second round of PCR reaction; Panel B: detection of tomato transcripts in rabbit tissues using RT-PCR followed by a second round of PCR. For the endogenous gene transcript controls, encoding rabbit β-globin and Rsp16, as well as positive controls using tomato total RNA (in panel B), only RT-PCR was performed. Blank: lane where no sample was loaded.

FIG. 6. Detection of tomato transcripts in the blood of rabbits after ingestion of ripened fruits in the diet. Rabbits of ˜1.8 kg were fed for 12 days with either normal chow (hay blocks+pellets) or a 1:1 mix (w/w) of diced tomato+chow. Due to lower fruit set for the HCVi lines, we have to pool all four lines for this construct while for the HIVi line 20B, there were only sufficient tomatoes for 10 days of feeding. Total RNA was prepare from each rabbit and used for cDNA synthesis using RT and random oligonucleotide or KLP primers. Transcripts were then assayed by qPCR using the TaqMan™ method. Panel A: detection of plastid 16S rRNA from tomato in the blood of rabbits post-ingestion (AACt values were calculated with β-globin as the endogenous reference transcript); Panel B: detection and quantification of tomato miRNA164 relative to endogenous 5.8S rRNA in blood; Panel C: detection and quantification of siRNAs in blood. WT: wild-type tomato fed control; other transgenic tomato lines are as indicated. Y-axis is in Log₁₀ scale for panels B and C to show the large dynamic range of the method.

FIG. 7: Stability and tissue distribution of tomato siRNAs in rabbits after ingestion of transgenic fruits. a) A mature rabbit (3.4 kg) was fed with HCVi tomatoes (a mixture of lines 11A1 and 14A3; FIG. 2) in a 1:1 mix (W/w) with normal rabbit chow. At day 14, no more tomatoes were fed to the animal and blood was drawn at 7, 14, and 28 days for transcript analysis. The ration of HCVi siRNA to the rabbit 5.8S rRNA is shown on a Log₁₀ scale. B) Detection of HCVi siRNA in tissues from rabbits after ingestion of transgenic tomatoes provided with the diet. Rabbits were either fed with wild-type (WT) tomatoes continuously for 28 days or supplemented with transgenic tomatoes (HCVi) for 14 days and then chow only until day 28. The animals were then sacrificed and brain (B), spleen (S) and liver (L) tissues removed for RNA preparation. Transcript analyses were performed by qRT-PCR as described above for rabbit 5.8S rRNA, tomato miRNA164 and for HCVi siRNAs. The ratio of mirRNA164 (blue bars) or HCVi siRNA (red bars) relative to the cognate 5.8S rRNA is shown on a Log₁₀ scale for the various samples. HCVi siRNAs are only detected in tissues of the HCVi-fed rabbits.

FIG. 8. Demonstration of efficacy of tomato fruit-derived sRNAs in targeting respective viral sequences. Left panel outlines the structure of the silencing detection vector using the humanized Renilla luciferase (hRLuc) as reporter while a humanized Firefly luciferase (hFFLuc) gene serves as an internal reference for normalization. Insertion of a single copy of the respective sequences used for our dsRNA binary vectors into a polylinker region between the stop codon of hRLuc and a synthetic PolyA addition signal generated the three vectors as indicated. pSV40: promoter from SV40; pTK: thymidine kinase promoter from HSV; tSV40: transcription terminator from SV40. Right panel: Human HepG2 cells were transfected with each of the vectors together with sRNA preparations from either wild-type (WT) or transgenic tomato fruits with the constructs NPi, HIVi or HCVi, as indicated. The ratios of Rluc/FFLuc activities obtained without plant sRNA addition (Mock) for each of the vectors are set as 100 for normalization of the other values. Cell extracts were prepared 2 days after transfection and used for luciferase assays after adjusting for protein concentrations. Percentages of reduction in hRluc activity compared to ‘Mock’ are also listed above the bars.

FIG. 9: Detection of small RNA (sRNA) uptake from tomato fruits in mice via ingestion. Two groups of 4 mice each are fed for 14 days with tomato juice extracts prepared from either wild-type (WT) or transgenic HCVi tomatoes (bottom panel) by the gavage method. Male mice (˜30 g each) were gavaged twice daily with ˜200 μL of tomato juice each time. 75-100 μL of blood were drawn from each mouse on day 0 (the day gavage began), day 14 (when gavage was stopped) and on day 18. Total RNA was prepared from each blood sample and the levels of endogenous mi16 (a mammalian microRNA used as an internal standard) tomato miRNA164 (blue bars) and HCVi siRNA (red bars) were quantified by RT-PCR. Titrations with synthetic mi16, miRNA164 and HCVi siRNA were used in the RT-PCR assays to create standard curves for generating accurate quantifications for each species of sRNAs.

### DETAILED DESCRIPTION OF THE INVENTION

Edible plants and plant products engineered to express functional RNAs (i.e. siRNA, miRNA, LncRNA, etc.) should be useful for modulating gene expression in mammals ingesting such products. For example, antiviral small interfering RNAs (siRNAs) have been shown to effect sequence specific cleavage, a phenomenon referred to in the literature as RNA silencing or interference (RNAi). siRNAs can be designed to target critical regions of viral transcripts or the RNAs in animals that produce proteins which are required for disease development such as pathogen susceptibility or cancer. Aside from RNAi, RNA molecules can also be used to trigger other cellular pathways that can have potential health benefits such as cell specific programmed cell death.

In addition to ingestion of edible fruits, other plant organs as well as processed plant products that retain the engineered RNA species should also be effective as an oral delivery vehicle for the RNA therapeutics. This approach can be used to advantage for viral prevention and treatment for Human Immunodeficiency Virus (Jacque et al. 2002), Hepatitis A and C Viruses (Kusov et al. 2006; Pan et al. 2009), Poliovirus (Gitlin et al. 2002) and Influenza Virus (Ge et al. 2003). In addition, other potential therapeutics targets such as mammalian factors involved in cancer metastasis, blood coagulation trigger tissue factors, membrane receptors involved in virus internalization, oncogenes—to name just a few examples—could be candidates for this strategy as treatment and/or prevention of disease as well. Thus, the present data focused on antiviral siRNAs should be viewed as examples of the types of therapeutically beneficial molecules that can be delivered using the compositions and methods of the present invention. The invention is not limited to these embodiments.

Current RNA therapeutics technology, which involves synthetic small RNA molecules, is costly in terms of nucleotide synthesis, storage, shipping and delivery. The common delivery route by injection is also undesirable, especially in cases where regular, repeated application is necessary. The use of edible transgenic plants and plant products as delivery vehicles for RNA therapeutics can provide a low-cost alternative approach that can be incorporated into the diet. This will also avoid the need for repeated injections, which is inconvenient as well as painful, in addition to increasing the chance of spreading of diseases or contracting new infections.

### Definitions:

The compositions and methods of the invention are useful for down modulation of RNA targets in mammalian cells. The terms “inhibit,” “inhibition,” “inhibiting”, “reduced”, “reduction” and the like as used herein refer to any decrease in the expression or function of a target gene product, including any relative decrement in expression or function up to and including complete abrogation of expression or function of the target gene product.

The term “expression” as used herein in the context of a gene product refers to the biosynthesis of that gene product, including the transcription and/or translation of the gene product. Inhibition of expression or function of a target gene product (i.e., a gene product of interest) can be in the context of a comparison between any two plants, for example, expression or function of a target gene product in a genetically altered plant versus the expression or function of that target gene product in a corresponding wild-type plant. Alternatively, inhibition of expression or function of the target gene product can be in the context of a comparison between plant cells, organelles, organs, tissues, or plant parts within the same plant or between plants, and includes comparisons between developmental or temporal stages within the same plant or between plants. Any method or composition that down-regulates expression of a target gene product, either at the level of transcription or translation, or down-regulates functional activity of the target gene product can be used to achieve inhibition of expression or function of the target gene product.

The term “inhibitory sequence” encompasses any polynucleotide or polypeptide sequence that is capable of inhibiting the expression of a target gene product, for example, at the level of transcription or translation, or which is capable of inhibiting the function of a target gene product. Exemplary constructs encoding such inhibitory sequences are disclosed herein.

When the phrase “capable of inhibiting” is used in the context of a polynucleotide inhibitory sequence, it is intended to mean that the inhibitory sequence itself exerts the inhibitory effect; or, where the inhibitory sequence encodes an inhibitory nucleotide molecule (for example, hairpin RNA, miRNA, or double-stranded RNA polynucleotides), or encodes an inhibitory polypeptide (i.e., a polypeptide that inhibits expression or function of the target gene product), following its transcription (for example, in the case of an inhibitory sequence encoding a hairpin RNA, miRNA, or double-stranded RNA polynucleotide) or its transcription and translation (in the case of an inhibitory sequence encoding an inhibitory polypeptide), the transcribed or translated product, respectively, exerts the inhibitory effect on the target gene product (i.e., inhibits expression or function of the target gene product).

Conversely, the terms “increase”, “increased, ” and “increasing” in the context of the methods of the present invention refer to any increase in the expression or function of a gene product, including any relative increment in expression or function.

In many instances the nucleotide sequences for use in the methods of the present invention, are provided in transcriptional units with for transcription in the plant of interest. A transcriptional unit is comprised generally of a promoter and a nucleotide sequence operably linked in the 3′ direction of the promoter, optionally with a terminator.

“Operably linked” refers to the functional linkage between a promoter and a second sequence, wherein the promoter sequence initiates and mediates transcription of the DNA sequence corresponding to the second sequence. The expression cassette will include 5′ and 3′ regulatory sequences operably linked to at least one of the sequences of the invention.

Generally, in the context of an over expression cassette, operably linked means that the nucleotide sequences being linked are contiguous and, where necessary to join two or more protein coding regions, contiguous and in the same reading frame. In the case where an expression cassette contains two or more protein coding regions joined in a contiguous manner in the same reading frame, the encoded polypeptide is herein defined as a “heterologous polypeptide” or a “chimeric polypeptide” or a “fusion polypeptide”. The cassette may additionally contain at least one additional coding sequence to be co-transformed into the organism. Alternatively, the additional coding sequence(s) can be provided on multiple expression cassettes.

The methods of transgenic expression can be used to decrease the level of at least one targeted viral sequence, following ingestion of transgenic tomatoes expressing an RNAi specific for the viral transcript. In cases where multiple molecules are required to suppress gene expression or viral replication the transgenic plants or fruits of the invention can comprise 1, 2, 3, 4 or 5 or more different therapeutically beneficial sRNA molecules. The methods of transgenic expression comprise transforming a plant cell with at least one expression cassette comprising a promoter that drives expression in the plant operably linked to at least one nucleotide sequence encoding an RNAi that inhibits production of the desired target protein(s) encoded either by viruses or in the animal. Methods for expressing transgenic genes in plants are well known in the art.

Plant transformants containing a desired genetic modification as a result of any of the above described methods can be selected by various methods known in the art. These methods include, but are not limited to, methods such as SDS-PAGE analysis, immunoblotting using antibodies which bind to the seed protein of interest, single nucleotide polymorphism (SNP) analysis, or assaying for the products of a reporter or marker gene, and the like.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. GUS is exemplified herein. The nucleic acid may be either RNA or DNA, linear or circular, single or double stranded, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The materials and methods set forth below are provided to facilitate the practice of the present invention.

### Methods for Constructing RNAi Vectors

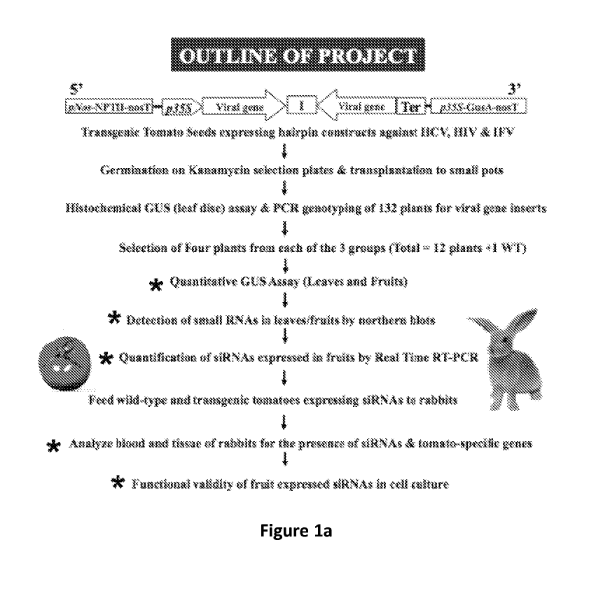

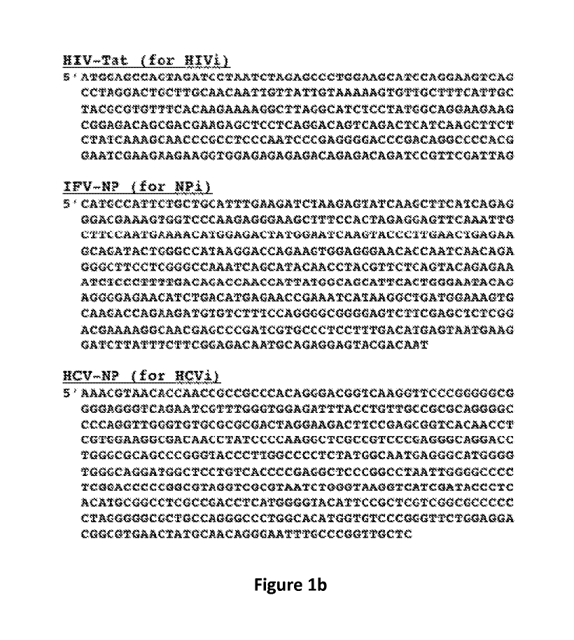

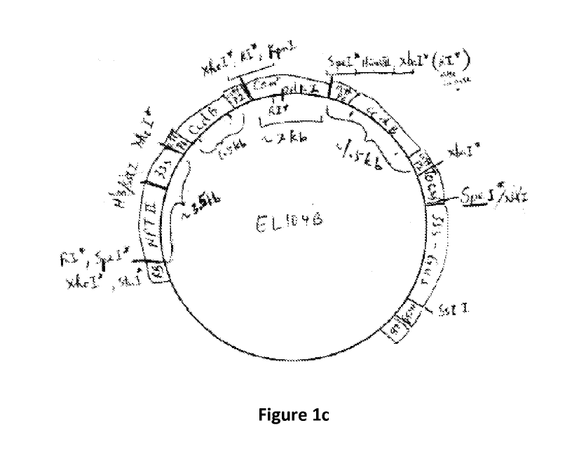

To facilitate the construction of double strand RNA (dsRNA)-producing vector for plant transformation, a Gateway Technology-based vector was constructed in the backbone of pBI121 (Lam E, unpublished result) using the recombinase targeting regions derived from the Hellsgate2 vector (Wesley et al. 2001). This binary vector, designated as EL420 (FIG. 1c), was then used to construct the vectors HIVi, NPi and HCVi for siRNAs targeting essential components of HIV, IFV and HCV, respectively, as described below and in FIG. 1.

### Methods Used to Transform Tomatoes

Cotyledon explants from sterile tomato (cv. Moneymaker) were used for transformation with the HIVi, NPi and HCVi vectors via Agrobacterium as described by McCormick (1991) and tissue culture conditions modified by Ruf et al. (2001). The skilled person is well aware of the variety of different methods currently available to introduce exogenous nucleic acids into plants and plant cells. These include without limitation, electroporation, biolistic delivery of DNA coated particles, protoplast fusion, transfection etc.

The following example is provided to facilitate practice of the present invention. It is not intended to limit the invention in any way.

### EXAMPLES

We have constructed binary vectors using 306 to 500 base pairs (bps) of essential regions from the influenza virus H1N1 (IFV), hepatitis C virus (HCV) and human immunodeficiency virus (HIV) to produce dsRNAs from these sequences in transgenic plants. 500 and 498 bps of the nucleoprotein (NP) encoding segments of the H1N1 and HCV were used respectively, while 306 bps of the Tat gene from HIV was used (FIG. 1b). The dsRNA transcripts thus produced in transgenic plants are known to undergo rapid processing into siRNAs to target specific sequences for cleavage. The vector is shown in FIG. 1c along a schematic flow chart of the steps taken to reduce the invention to practice (FIG. 1a). The respective viral sequences were first cloned into our gene suppression vector as inverted repeats that are separated by an intron from the Pdk gene of Flaveria containing a chloramphenicol resistance marker, which is spliced out in plant cells after the production of the nascent transcript in the nucleus (Smith et al. 2000; Wesley et al. 2001). We also incorporated a reporter gene (GusA) in our transformation cassette that affords simple and rapid detection of transgene insertion in our transgenic plants. This reporter encodes a bacterial β-glucuronidase (GUS) that facilitates both histochemical detection and biochemical quantification of transgene activity in the insertion locus. Using these vectors, transgenic tomato lines were constructed using published protocols via Agrobacterium (McCormick 1991; Ruf et al. 2001) in the laboratory of Prof Ralph Bock at the Max-Planck Institute of Molecular Plant Physiology in Postdam/Golm, Germany. Due to potential silencing of the various insertion constructs, we first screened for transgenic plants by selection on antibiotic-containing plates using the kanamycin resistance marker (NPTII) on our vector. A total of 132 transgenic tomato plants were thus selected from progeny of 26 independent lines of transgenic tomato and transplanted to soil. We further tested these tomato plants for transgene activity by rapid leaf tissue histochemical staining using X-Gluc as well as by PCR genotyping using primers specific for each of the viral sequences. Although >90% of the selected plants were verified to contain the expected viral transgene by PCR genotyping, only about half of these plants showed detectable blue staining when leaf tissues were incubated overnight with the X-Gluc substrate (data not shown). The small percentage of the lines that failed to contain the viral gene fragment may have resulted from the partial loss of the integrating vector during these transformation events. Lines not showing GUS expression likely result from transgene silencing due to mechanisms triggered by complex integration. We chose 4 plants from each of the 3 constructs with different levels of GUS expression in order to propagate a manageable number of plants for bulking of fruits and to carry out further analyses as outlined in FIG. 1.

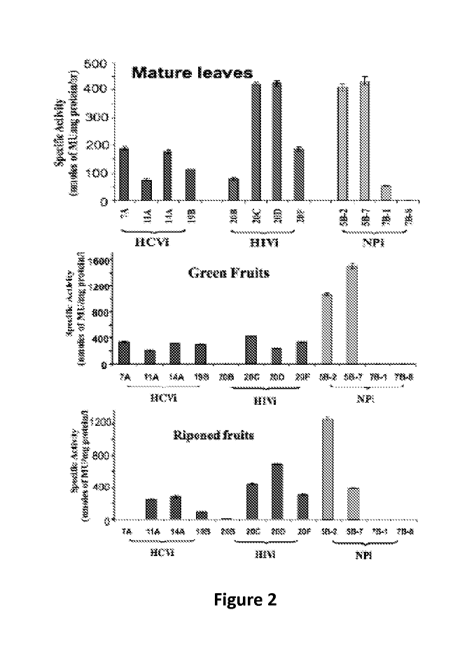

We carried out quantitative analysis of GUS expression in these 12 transgenic tomato lines in leaves of mature plants, as well as from green and ripened fruits (FIG. 2). The 3 types of transgenic plant lines were designated as HCVi (HCV interfering), HIVi (HIV interfering) and NPi (IFV interfering). Among these plants we readily observed some lines showing tissue-specific silencing of GUS in the fruits (i.e. line 7A of HCVi, line 20B of HIVi, and to some extent, lines 5B-7 and 7B-1 of NPi). Interestingly, for two of the NPi lines, 5B-2 and 5B-7, we observed an increase of about 4 fold in GUS expression when activities in green fruits vs. mature leaves are compared.

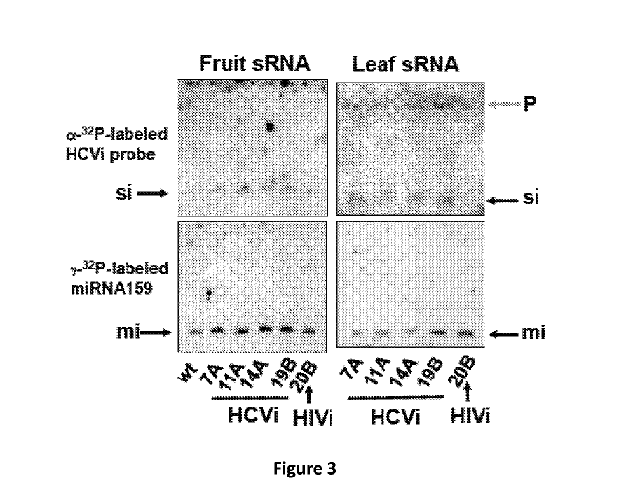

After this series of analyses, we propagated these tomato lines asexually by stem cuttings and transplantation of the new shoots. At the same time, we also worked to detect the production of the target siRNA repertoires for the respective viral sequences in the tomato lines. First, we worked to detect the siRNAs produced in the leaf and fruit tissues of the transgenic plants. We followed published protocols to enrich for sRNAs in our nucleic acid purification method, followed by polyacrylamide gel electrophoresis (PAGE) separation and northern blot analyses. FIG. 3 shows that we were able to detect the expected ˜21 nucleotide siRNA species in leaf and fruit tissues from our transgenic tomato plants for the HCVi lines using ³²P-labeled HCVi probes. This HCVi probe did not cross-hybridize with sRNA from one of the HIVi lines as expected (shown here on the right lane).

Surprisingly, similar levels of siRNAs were detected for the 4 NPi lines (data not shown) as in the 4 HCVi lines although two of these lines (7B-1, 7B-8) showed very low GUS expression in ripened fruits (FIG. 2). This result suggests that the relative expression between the GUS gene and the dsRNA-generating cassette in our binary vector are not necessarily correlated. Using a radiolabeled oligonucleotide that hybridizes to tomato miRNA159, we could detect signals from all the sRNA samples in the two blots after stripping away the HCVi probe. As expected, the miRNA and siRNA bands have very similar sizes and thus relative mobility on PAGE analyses. No significant signal for siRNAs can be detected in the HIVi plants under our conditions for northern blots although the miRNA159 control transcript can be readily seen in the sRNA preparations as in the other lines (data not shown). Thus, it appears that the HIVi transgenic tomato lines expressed lower levels of siRNAs than the HCVi and NPi lines. Additional transgenic lines with the HIVi construct can be generated to obtain higher expressor lines.

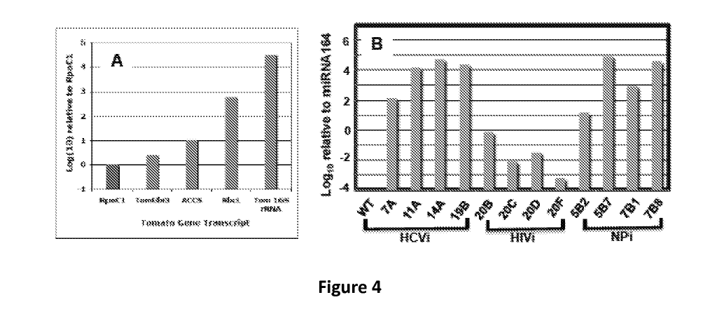

To quantify the small RNAs such as miRNAs and siRNAs in tomato fruits with high sensitivity and specificity, we adapted the “Key-Like-Primer” (KLP)-TaqMan technology recently published by Yang et al. (Plant Biotech. J. [2009] 7:1-10) for the quantitative and specific detection of siRNAs. This method has been shown to be capable of detecting miRNAs and siRNAs at levels as low as 10 copies per cell. Importantly, this technique is amenable to the use of total RNA and thus obviates the need to use siRNA preparations that are more tedious to prepare. To aid our effort in detecting and quantifying various tomato RNA species in animal tissues and blood after ingestion, we also designed and synthesized TaqMan PCR probes for various nuclear- and plastid-encoded genes of tomato. The data from their use in transcript quantification using tomato fruit RNAs prepared from wild-type (WT) or transgenic lines are shown in FIG. 4.

The relative levels of transcript for the five protein- and rRNA-encoding genes are in the order of 16S rRNA>RbcL>ACCS>TomUbi3>RpoC1 (panel A, FIG. 4). Both the 16S rRNA and RbcL are highly expressed plastid transcripts while RpoC1 is a subunit of the plastid RNA polymerase that is expressed at very low levels. The two tomato nuclear genes ACCS and Ubi3 are expressed at levels that are ˜100 times lower than the RbcL transcript in the fruit. To measure the level of siRNAs produced in our transgenic tomato fruits, we used miRNA164 as an internal reference. This was chosen since this miRNA164, as opposed to miRNA159, is more highly expressed in the tomato fruit (Itaya et al. [2008] BBA 1779: 99-107). Multiple KLP oligonucleotides were designed and tested for each of the target sRNAs until their specificity and efficacy are verified. In FIG. 4 (panel B), the siRNAs produced in the HCVi, HIVi and NPi transgenic lines are quantified with the respective KLP probes. These KLP probes do not detect any appreciable amplification signals in WT tomato fruits and they also are specific for each of the viral sequence targets (data not shown). Our data show that while the highest HIVi expressor (line 20B) produced siRNAs to the level of miRNA164 at steady state, our transgenic lines for HCVi and NPi can accumulate levels of siRNAs that are 4 to 5 orders of magnitude higher than this endogenous miRNA in the tomato fruit.

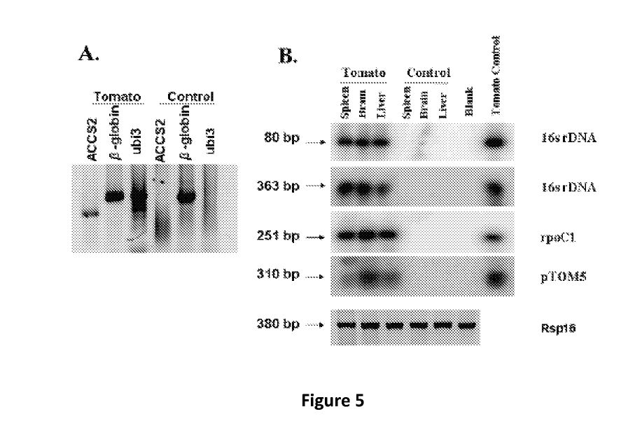

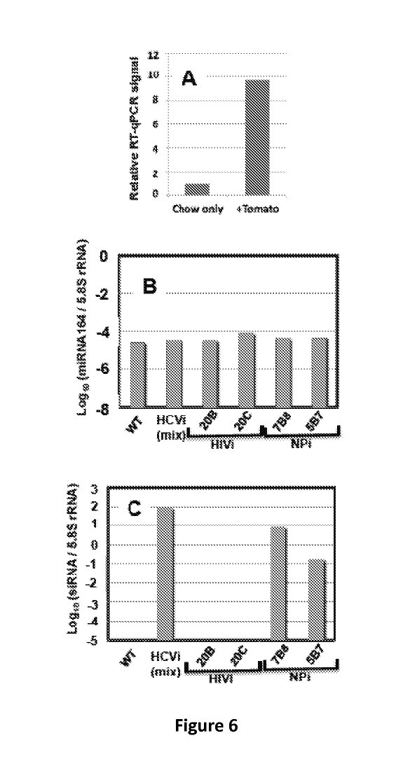

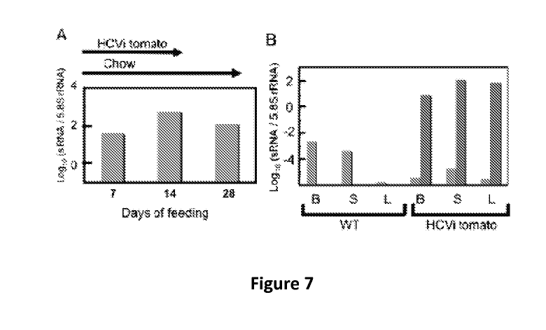

In the case of C. elegans, dsRNA are expressed in E. coli bacteria which in turn are used as food for the nematode. The RNAi signal can then persist to the next generation after the initial feeding to silence expression of the targeted gene. The mammalian digestive system is very different from that of nematodes, however the potential benefit from such a simple delivery route for RNAi triggers warrants its examination. To examine the question of whether tomato-derived RNAs can in fact survive the mammalian digestive system and make it to the bloodstream and organs, we have performed feeding studies with rabbits that have been fed a simple tomato diet for a week before blood and tissues are drawn and analyzed for the presence of tomato-specific transcripts. In FIGS. 5a and 5b, our data showed evidence for the presence of tomato RNAs derived from both the nuclear and plastid genomes. However, the levels of these tomato RNAs are apparently very low in the rabbits, since it required multiple rounds of PCR amplifications for their detection by endpoint PCR assays. Although very sensitive, this approach, involving sequential rounds of PCR amplification makes it very difficult to quantify the levels of the transcripts. Using the RT-qPCR strategy of Yang et al. (2009), we quantified the levels of tomato fruit transcripts in rabbits after their ingestion, with special emphasis on the siRNA from the transgenic tomato fruits (FIG. 6). Among the 5 transcript targets shown in FIG. 4A, the most abundant 16S rRNA from tomato plastids is the one that can be detected routinely in the blood of rabbits after feeding. FIG. 6A shows that we can detect this transcript without pre-amplification in total RNA isolated from blood and it shows at least a 10-fold higher levels than the background seen with the mock-treated (chow only) rabbit. Among the other 4 transcripts, we can also routinely detect RbcL but usually at lower levels than 16S rRNA, requiring more PCR amplification cycles for detection while the other 3 are not seen with confidence using the TaqMan method. For miRNA164, however, our KLP-Taqman method worked well to detect this transcript in rabbit blood. FIG. 6B shows its detection in various tomato (both WT and transgenic) fed rabbits. No significant signals are detected with blood from mock-treated rabbits under our conditions (data not shown). Importantly, our data shows that there is relatively small variance (2 to 3 fold) in the amount of miRNA164 detected among six different rabbits that were fed different lines of tomato fruits in parallel. Relative to the rabbit 5.8S rRNA transcript, which we also measured using the KLP-TaqMan method, we estimated that the tomato miRNA164 is present at levels that are at least ˜10⁴ times lower than this cognate RNA reference. Remarkably, quantifying the siRNAs in these RNA samples revealed that we could readily detect the uptake of the specific siRNAs produced in the HCVi and NPi lines. In the HCVi tomato-fed rabbit, the level of siRNAs was estimated at almost 100× that of the endogenous 5.8S rRNA (FIG. 6C). It is perhaps not surprising that we failed to detect the siRNAs in rabbits fed with HIVi tomato lines since these siRNAs are present at levels that are 4 to 5 orders of magnitude lower than with the HCVi and NPi lines (FIG. 4). For the two NPi tomato-fed rabbits, siRNAs are detected at similar levels to that of the 5.8S rRNA reference. In summary, our data demonstrate that our vectors and approach can achieve good levels of siRNA production in stably transformed tomato fruits. Furthermore, the sRNAs in fruits (e.g. miRNAs and siRNAs) can be taken up efficiently into the circulatory system of mammals after their ingestion as part of the diet. Steady-state levels of siRNAs approaching that of an abundant cognate rRNA (i.e. 5.8S rRNA) can be achieved by this oral delivery technology. We can then determine the stability and tissue accessibility of the plant-derived siRNAs that were taken up into the rabbit's circulatory system after ingestion of tomato fruits. FIG. 7A shows an experiment in which after no tomato was provided to the rabbit after feeding for 2 weeks with our transgenic HCVi tomatoes. The levels of HCVi siRNA remained relatively high even after two weeks of feeding on rabbit chow only, thus showing that the siRNAs taken up can be stable for up to two weeks in the blood. Furthermore, we analyzed the tissues (brain, spleen and liver) of the rabbit after sacrificing it at the end of the experiment and we can readily detect them in these organs, thus demonstrating their ability to be taken up into these tissues (FIG. 7B). The relatively high levels of the siRNAs detected, as compared to that observed in the blood of the animal, indicates that the RNAs detected in these organs is unlikely to derive from the small amount of blood that may be present inside these organs.

Viruses such as hepatitis C virus (HCV), avian influenza (H5N1) and the human immunodeficiency virus (HIV) pose difficult targets for traditional therapies and preventive measures. Their rapid mutation and recombination rates make monotherapeutics involving single targets ineffective in the long term. Although RNA interference (RNAi) triggered by administration or expression of small double-stranded RNAs (dsRNAs) of 21-25 nucleotides targeting essential components of various viruses has been shown to be effective in suppressing their replication in host cells, spontaneous mutations in the targeted virus enable the virus to escape this repression. In addition, the ultimate deployment of RNAi as an antiviral therapeutics in the developing world will likely be hampered by the economics involved.

As demonstrated herein and elsewhere, plant cells tolerate long strand dsRNAs from several hundred up to several thousand bases and thus can produce a large repertoire of siRNAs from a single transcription unit. Previously, we have demonstrated that siRNAs (small interfering RNAs) targeting a 400 nucleotide conserved region of the influenza NS1 protein can be constitutively produced in transgenic tobacco and these plant-derived siRNAs are effective in virus suppression in transfection studies involving mammalian cells (Zhou et al. FEBS Lett., 2004). This strategy can be easily scaled up to express several kilobases (kbs) of sequences that would contain multiple viral genome targets for combinatorial silencing. Thus plants are excellent natural “factories” to produce complex RNAi triggers economically on an agricultural scale.

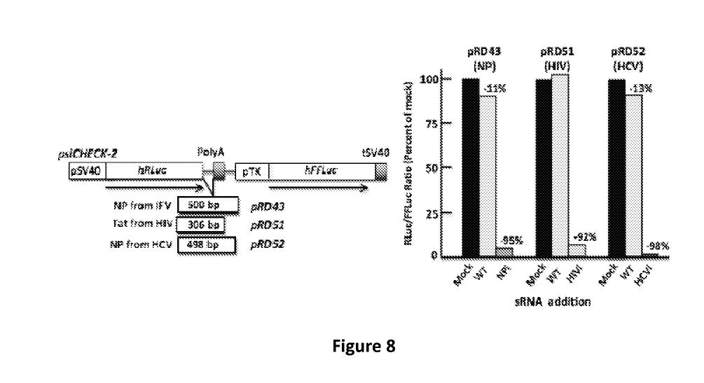

In additional experiments, we demonstrate that tomato-expressed siRNAs down modulate the appropriate viral sequences in mammalian cells. In addition to detecting the production of siRNA repertoires against IFV (NPi), HCV (HCVi) and HIV (HIVi) in tomato fruits, we wanted to verify and quantify the efficacy of these plant-derived siRNAs in eliciting the suppression of the respective viral sequences. Such RNAi effects can be readily assayed in animal cell culture using a convenient assay that employs the psiCHECK-2™ vector system (Promega Co., WI). By using a dual luciferase expression cassette that allows the simultaneous quantification of the test gene vs. a common reference gene, this system enables facile normalization of the gene suppression that may result from RNAi suppression via an inserted sequence. Three different psiCHECK-2 derived constructs (pRD43, pRD51 and pRD52) with the viral sequences that were used to construct the binary vectors shown in FIG. 1 were generated (FIG. 8). Specific targeted cleavage at these viral sequences by siRNA-mediated RNAi is predicted to lower expression of the hRLuc gene while no effect will be expected for the neighboring hFFLuc gene. These three vectors were then each tested in human HepG2 cells by transfection together with siRNA preparations from either WT or transgenic tomato fruits that expressed the respective siRNAs. Remarkably, all three sRNA preparations from the transgenic fruits were able to show robust suppression (>90%) of the test gene hRLuc with little non-specific effect seen on the hFFLuc gene expression (data not shown). Importantly, siRNA preparations from WT fruits with comparable quality as judged by similar concentrations of micRNA164 showed no significant effects.

These data demonstrate the high degree of specificity and efficacy of the three sets of siRNAs that our transgenic fruits produced. The degree of specific gene suppression observed in our assay is especially impressive since previous work with synthetic siRNA or shRNA designed for a specific site on target genes are usually not very effective, with an average of only 1 in 5 of the sites selected showing efficient gene silencing of 80% or more (Kapadia et al. [2003] PNAS 100: 2014-8; Hohjoh [2002] FEBS Lett. 521: 195-9). This can be explained by the fact that in generating a collection of ˜21 nt siRNAs from our viral sequences, our plant expression system likely produces a repertoire of 15 to 25 distinct siRNA species from each of our transgenes in the different tomato lines. The increase in potential target site numbers for the transcript in question thus can contribute to the more robust suppression observed. Our observations demonstrated that fruit-derived siRNA repertoires could direct efficient and specific RNAi against transcripts encoding critical components of animal viral pathogens.

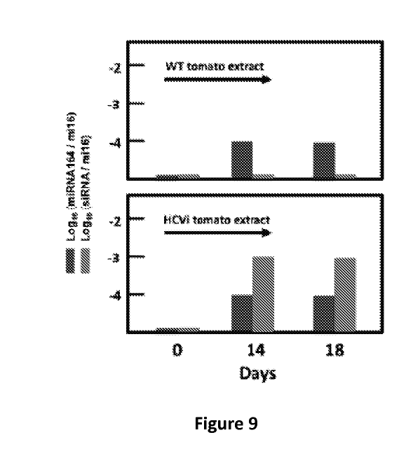

Lastly, we also tested the generality of this uptake phenomenon by examining the ability of plant miRNA and transgenically-produced siRNAs to be taken up in mice via the GI tract. FIG. 9 shows that both tomato miRNA164 as well as anti-viral HCVi siRNAs can be shown to accumulate in the circulatory system of mice after their ingestion via gavage. Furthermore, these sRNAs are found to be stable for at least 4 days after termination of their feeding to the animal. These observations suggest that the results we obtained for rabbits are likely applicable to other mammals.

In a recent study, mature plant miRNA has been reported to be especially stable in the GI tract of mammals, likely due to the specific methylation that is found in sRNAs of plants but not usually found in animal sRNAs (Zhang et al. 2012). Plant siRNAs are also methylated in a similar fashion and thus are expected to be similarly protected. In the data reported, the rice miRNA168a was found to be incorporated into the mouse RISC complex (based on immunoprecipitation studies with anti-AGO2 antiserum) after uptake in mice and was functional to suppress its target in the animal's cells via translational inhibition. This crucial data provides support for the expectation that the plant-derived sRNAs will be functional after their uptake into the animal's system. Finally, the rice miRNAs under investigation were found to be accessible to various mice tissues, as we have shown for rabbits.

### REFERENCE LIST

Ge et al. [2003] PNAS 100: 2718-2723
Gitlin et al. [2002] Nature 418: 430-434
Hohjoh [2002] FEBS Lett. 521: 195-9
Itaya et al. [2008] BBA 1779: 99-107
Jacque et al. [2002] Nature 418: 435-438
Kapadia et al. [2003] PNAS 100: 2014-8
Kusov et al. [2006] J. of Virology 80: 5599-5610
McCormick, S. [1991] Plant Tissue Culture Manual B6:1-9.
Pan et al. [2009] J. Mol. Med. 87: 713-722
Ruf, S., Hermann, M., Berger, I. J., Carrer, H., Bock, R. [2001] Nat. Biotechnol. 19:870-875.
Smith et al. [2000] Nature 407:319-20.
Wesley et al. [2001] The Plant Journal 27:581-590.
Yang et al. [2009] Plant Biotech. J. 7:1-10
Zhou et al. [2004] FEBS lett.,—PDF attached to filing
Zhang et al. [2012] Cell Research 22: 107-126

While the invention has been described in detail and with reference to specific examples thereof, it will be apparent to one skilled in the art that various changes and modifications can be made therein without departing from the spirit and scope thereof.

What is claimed is:

1. A transgenic plant or fruit comprising a nucleic acid construct encoding at least one inhibitory RNA molecule which is effective to inhibit expression of a target nucleic acid upon ingestion of said plant or fruit or an edible portion thereof when consumed by a test subject.

2. The transgenic plant or fruit of claim 1, selected from the group consisting of tomato, tobacco, carrot, lettuce, potato, rice, corn, cucumber and zucchini.

3. The transgenic plant or fruit of claim 2 which is a tomato.

4. The transgenic tomato of claim 3, wherein said inhibitory RNA molecule is effective to inhibit replication and infection of mammalian cells by human hepatitis C virus, in a mammal ingesting said tomato.

5. The transgenic tomato of claim 1, wherein said inhibitory RNA molecule is effective to inhibit replication and infection of mammalian cells by human immunodeficiency virus, in a mammal ingesting said tomato.

6. The transgenic tomato of claim 1, wherein said inhibitory RNA molecule is effective to inhibit replication and infection of mammalian cells by influenza virus, in a mammal ingesting said tomato.

7. The transgenic tomato plant as claimed in claim 3 which comprises a nucleic acid construct selected from the group of constructs shown in FIG. b.

8. A method for inhibiting viral infection in a mammal, comprising ingestion of an effective amount of transgenic tomato by said mammal, said tomato expressing the nucleic acid construct of claim 1, wherein expression of said inhibitory RNA from said construct is effective to inhibit viral infection and/or replication in said mammal.

9. The method of claim 8, wherein said viral infection is caused by HCV.

10. The method of claim 8, wherein said viral infection is caused by influenza.

11. The method of claim 8, wherein said viral infection is caused by HCV.

12. Seeds obtained from the plant or fruit of claim 1.

13. Seeds obtained from the transgenic tomato of claim 3.


SEQUENCE LISTING (3 sequences)

SEQ ID NO: 1 - DNA, 306 bp, Artificial Sequence
  Synthetic sequence
atggagccagtagatcctaatctagagccctggaagcatccaggaagtcagcctaggactgcttgcaacaattgttattgtaaaaactgttgctttcattgctacgcgtgtttcacaagaaaaggcttaggcatctcctatggcaggaagaagcggagacagcgacgaagagctcctcaggacagtcacactcatcaagcttctctatcaaagcaacccgcctcccaatcccgaggggacccgacaggccccacggaatcgaagaagaaggtggagagagagacagagacagatccgttcgattag

SEQ ID NO: 2 - DNA, 500 bp, Artificial Sequence
  Synthetic sequence
catgccattctgctgcatttgaagatctaagagtatcaagcttcatcagagggacgaaagtggtcccaagagggaagctttccactagaggagttcaaattgcttccaatgaaaacatggagactatggaatcaagtacccttgaactgagaagcagatactgggccataaggaccagaagtggagggaacaccaatcaacagagggcttcctcgggccaaatcagcatacaacctacgttctcagtacagagaaatctcccttttgacagaccaaccattatggcagcattcactgggaatacagaggggagaacatctgacatgagaaccgaaatcataaggctgatggaaagtgcaagaccagaagatgtgtctttccaggggcggggactcttcgagctctcggacgaaaaggcaacgagcccgatcgtgccctcctttcacatgagtaatgaaggatcttatttcttcggagacaatgcagaggagtaccacaat

SEQ ID NO: 3 - DNA, 498 bp, Artificial Sequence
  Synthetic sequence
aaacgtaacaccaaccgccgcccacagggacggtcaaggttcccgggggcggggagggtcagaatcgtttgggtggagatttacctcttgccgcgcaggggccccaggttgggtgtgcgcgcgactaggaagacttccgagcggtcacaacctcgtggaaggcgacaacctatccccaaggctcgccgtcccgagggcaggacctgggcgcagcccgggtacccttggcccctctatggcaatgagggcatggggtgggcaggatggctcctgtcaccccgaggctcccgccctaattggggcccctcggacccccggcgtaggtcgcgtaatctgggtaacgtcatcgataccctcacatgcggcctcgccgacctcatggggtacattcccctcgtcggcgcccccctagggggcgctgccagggccctggcacatggtgtcccgggttctggaggacggcgtgaactatgcaacagggaatttgcccggttcctc